(19) United States
(12) Patent Application Publication
(10) Pub. No.: US 2008/0058612 A1
(43) Pub. Date: Mar. 6, 2008

(54) MEDICAL INFORMATION SYSTEM

(76) Inventors: Shigeharu Ohyu, Yaita-shi (JP); Yasuo Sakurai, Otawara-shi (JP); Satoshi Ikeda, Yaita-shi (JP); Kousuke Sakaue, Nasushiobara-shi (JP); Hitoshi Yamagata, Otawara-shi (JP); Yoshihisa Nakada, Shioya-gun (JP); Toshio Tanaka, Utsunomiya-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/849,773

(22) Filed: Sep. 4, 2007

(30) Foreign Application Priority Data
Sep. 5, 2006 (JP) 2006-240329

(51) Int. Cl. A61B 5/00; G06Q 50/00

(52) U.S. Cl. 600/300; 705/2

(57) ABSTRACT

A medical information system includes a measurement unit that measures at least the positions of medical instruments and biomedical information, a medical act identification unit that decides the types of performed medical acts and the time instants of performance, at which the medical acts are performed, on the basis of the measured positions of the medical instruments and the measured biomedical information, and a medical stage identification unit that decides the types of medical stages, into which a flow of steps starting with a step of preparing for surgery and ending with a step of completing the surgery is divided in terms of the purpose of work, and the time instants of the medical stages on the basis of the decided types of medical acts, the decided time instants of performance, the other types of medical stages that have already been decided, and the time instants of the other types of medical stages that have already been decided.

### CROSS-REFERENCE TO RELATED APPLICATIONS

This application is based upon and claims the benefit of priority from prior Japanese Patent Application No. 2006-240329, filed Sep. 5, 2006, the entire contents of which are incorporated herein by reference.

### BACKGROUND OF THE INVENTION

1. Field of the Invention

The present invention relates to a medical information system that records various medical acts performed during a period from the instant of preparing for surgery to the instant of completing the surgery.

2. Description of the Related Art

Medical information systems having the ability to detect the fact that a medical act has been performed and to record information on the medical act together with information on the time instant have been proposed. Although some of the systems have the ability to decide the type of process being under way, a method of collating acquired information with a table in which the features of processes are recorded, and deciding the type of process and the length thereof is generally implemented in the systems. This poses a problem in that only a process whose type and length can be decided based merely on acquired information can be identified. In an actual surgical process, nearly identical acts often have different purposes according to the relationships to preceding and succeeding processes. Therefore, the type of process and the length thereof cannot be often decided unless consideration is taken into the relationships to the preceding and succeeding processes. A process cannot be effectively identified based merely on acquired information.

Moreover, a system that records pieces of information on medical acts, which are acquired time-sequentially during surgery, together with pieces of information on time instants, and reads information after the completion of the surgery has been proposed. In the system, whether a predetermined condition is established relative to a detected signal is decided. When the predetermined condition is established, a cue is assigned to a signal detected at that time. For reading, the cue is referenced in order to retrieve the record.

The cue is useful in reading a record. However, since the cue is assigned based on a detected signal alone, the cue may be assigned to similar acts whose purposes are different from each other. This poses a problem in that information on a cue useful in reading a record cannot be provided. Moreover, acquired pieces of information may be displayed comprehensively.

However, what information has to be provided depends on a surgical stage. When an attempt is made to comprehensively display pieces of information in the same style, all data items have to be displayed. Consequently, an enormous information providing means becomes necessary. This makes it hard to acquire specific information from display. For details, refer to JP-A-2004-280455.

### SUMMARY OF THE INVENTION

An object of the present invention is to identify various medical stages of surgery.

A medical information system in accordance with an aspect of the present invention includes: a measurement unit that measures at least the positions of medical instruments and biomedical information; a medical act identification unit that decides the types of performed medical acts and the time instants of performance, at which the medical acts are performed, on the basis of the measured positions of medical instruments and the measured biomedical information; and a medical stage identification unit that decides the types of medical stages, into which a flow of steps starting with a step of preparing for surgery and ending with a step of completing the surgery is classified in terms of the purpose of work, and the time instants of the medical stages on the basis of the decided types of medical acts, the decided time instants of performance, the other types of medical stages that have already been decided, and the time instants of the other types of medical stages that have already been decided.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the preferred embodiments given below, serve to explain the principles of the invention.

FIG. 1 shows the configuration of a medical information system in accordance with an embodiment of the present invention;

FIG. 2 shows an example of application of the medical information system shown in FIG. 1 to anesthesia;

FIG. 3 shows examples of positions at which an IC tag reader shown in FIG. 2 may be disposed;

FIG. 4 is a flowchart describing medical stage identification to be performed according to the embodiment;

FIG. 5 lists rules for, for example, identifying an anesthesia introduction stage and relevant actions according to the embodiment;

FIG. 6 lists items to be displayed in the anesthesia introduction stage listed in FIG. 5;

FIG. 7 lists items to be entered by an anesthesiologist for the purpose of deciding whether the anesthesia introduction stage has been terminated (anesthesia has been completed);

FIG. 8 lists items to be displayed on a knee board in the anesthesia introduction stage listed in FIG. 5;

FIG. 9 shows actions relevant to a hemostasis medical stage to be executed according to the embodiment;

FIG. 10 shows actions relevant to an incision medical stage to be performed according to the embodiment;

FIG. 11 shows a cerebral spatula fixture to be employed in an exclusion medical stage according to the embodiment;

FIG. 12 shows a cerebral spatula to be employed in the exclusion stage according to the embodiment;

FIG. 13 shows an example of an anesthetic recording screen image to be displayed according to the embodiment; and

FIG. 14 shows the relationship between a craniotomy medical stage and a search medical stage employed in the embodiment.

### DETAILED DESCRIPTION OF THE INVENTION

Referring to the drawings, an embodiment of a medical information system in accordance with the present invention will be described below.

In the present embodiment, a medical act as well as a medical stage is an object of identification. The medical stage is identified based on the type of medical act and the time instant of performance thereof that are decided based on the positions of medical instruments and biomedical information, and the temporal relationships to the other preceding and succeeding medical stages that have already been identified. When a medical stage is identified, a repetitive medical act such as administration of a drug or the like can be discriminated based on the medical stage. In addition, advantages described below are provided.
When records are read, a feature for selecting a time instant according to a purpose can be provided.
Warning is given depending on a medical stage. Warning can be given based on a practical situation.
A criterion dependent on a medical stage can be introduced. An incident can be extracted more highly precisely. The incident refers to an overt or latent accidental factor (including a small accident and a trivial event that cannot be recognized as an accident) occurring during a medical act that has a possibility of triggering a medical accident or a significant incident directly linked to the medical accident. Examples of the incident are a shortage in workforce, a change of workers, a delay in a work start time instant, hastening of a work start time instant, a longer work time than a scheduled time, a shortage in a work time, use of an erroneous drug (in a kind of drug, a brand, a quantity, a sequence, or the like), use of an erroneous machine or instrument (in a type, a sequence, a magnitude, an operating time, or the like), and an abnormality in a blood loss, a blood pressure, a heart rate, a hematic component, or an anesthetic depth.
A medical act or a procedure can be effectively analyzed.

Moreover, based on a medical stage identified according to the present embodiment, appropriate surgical aid information can be selected and displayed in each stage of surgery. Moreover, a kind of information that should be entered in each stage of surgery can be notified, and entry of appropriate information can be prompted. Further, the inclusion of a warning deciding means dependent on a medical stage makes it possible to give warning more appropriately.

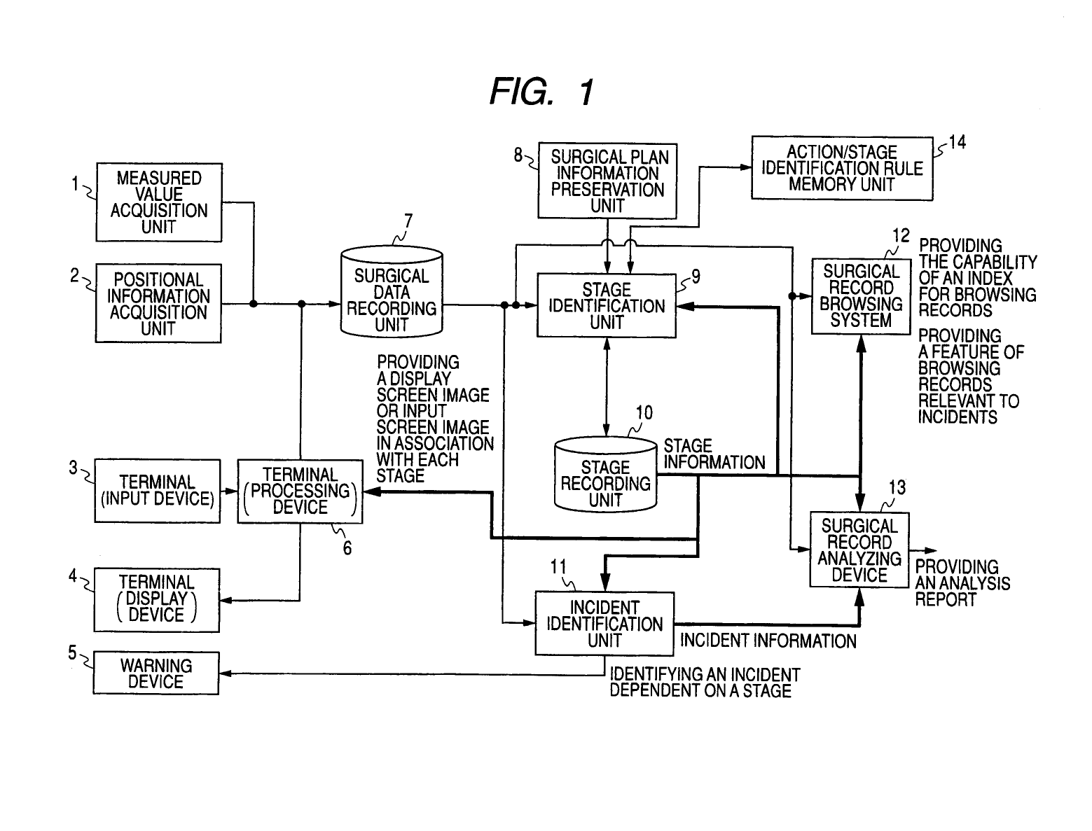

FIG. 1 shows the configuration of a medical information system in accordance with the embodiment. A measured value acquisition unit 1 acquires together with a measurement time instant a measured value of biomedical information such as a heart rate, a breathing rate, or a blood pressure from a sensor that is not shown. The acquired biomedical information is recorded in a surgical data recording unit 7. A positional information acquisition unit 2 that acquires the positions of medical instruments acquires the positions of various medical instruments employed in surgery, the users of the medical instruments, the use time instants of the medical instruments, the operating state of medical equipment, and the positions of medical act performers. The acquired pieces of information on the positions of the medical instructions and others are recorded in the surgical data recording unit 7.

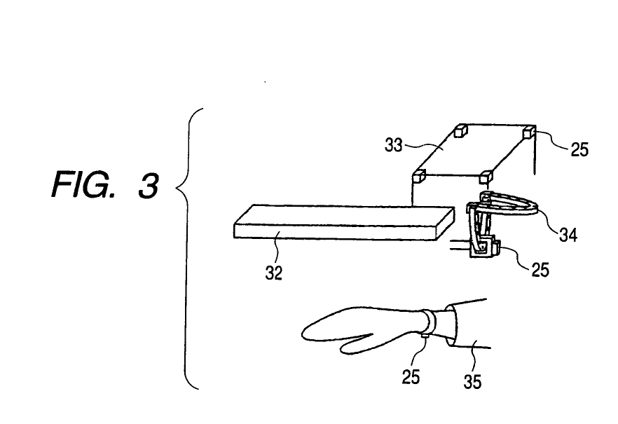

Typically, as shown in FIG. 3, an IC tag and an IC tag reader are used to detect the position of a medical instrument. For example, the IC tag is attached to a surgical instrument such as a knife, forceps, shears, bipolar forceps, robot forceps, a sheet of gauge, an infusion set, a catheter, or an endoscope, or medical equipment such as a diagnostic ultrasound system. Moreover, an IC tag reader 25 is attached to an operating table 32, a head brace 34, an instrument stage 33, a trash bin, an entrance of an operating room, a shelf in the operating room, or a chair in the operating room. When the IC tag reader 25 is worn on a surgeon's arm 35, whether the surgeon is holding a medical instrument can be detected.

A terminal serving as a man-machine interface of the medical information system includes, aside from a processing device 6, an input device 3, a display device 4, and a warning device 5 including a warning lamp and a loudspeaker. The surgical data recording unit 7 records biomedical information acquired by the measured value acquisition unit 1 and pieces of information on the positions of medical instruments and others acquired by the positional information acquisition unit 2. A surgical plan information preservation unit 8 preserves information on a surgical plan produced by a surgical planning system that is not shown. In surgical planning, a procedure of multiple medical stages, a procedure of multiple medical acts included in each medical stage, persons in charge, and a drug and a medical instrument employed for each medical act are determined.

In an action/stage identification rule memory unit 14, multiple rules based on which defined types of medical acts or actions and time instants of performance are decided are stored. Moreover, in the action/stage identification rule memory unit 14, multiple rules based on which defined types of medical stages and time instants of performance are decided are stored.

Under the medical act identification rules stored in the action/stage identification rule memory unit 14, a stage identification unit 9 decides the type of performed medical act and the time instant of performance on the basis of the biomedical information acquired by the measured value acquisition unit 1, and the positions of medical instruments acquired by the positional information acquisition unit 2, the users of the medical instruments, the use time instants of the medical instruments, the operating state of medical equipment, the positions of medical act performers, and the surgical plan. Moreover, under the stage identification rules stored in the action/stage identification rule memory unit 14, the stage identification unit 9 decides the type of medical stage and the time instant of performance (length of performance) on the basis of decided medical acts, a combination of medical acts, and the temporal relationships to other preceding and succeeding medical stages that have already been identified. A stage recording unit 10 records data items concerning the decided types of medical acts, the time instants of the medical acts, the decided type of medical stage, and the time instant of the medical stage.

The decided type of medical stage and the decided time instant thereof are used for identification of an incident to be performed by an incident identification unit 11 or for calculation of a predicted occurrence-of-accident value. Moreover, a surgical record browsing system 12 uses the decided type of medical stage and the decided time instant thereof as a record index. Moreover, a surgical record analyzing device 13 uses the decided type of medical stage and the decided time instant thereof for analysis. The surgical record browsing system 12 is a software system that records an image or a picture produced by an X-ray imaging apparatus or any other medical equipment, and records voice of a patient or a physician obtained through a microphone and a picture thereof formed by a camera.

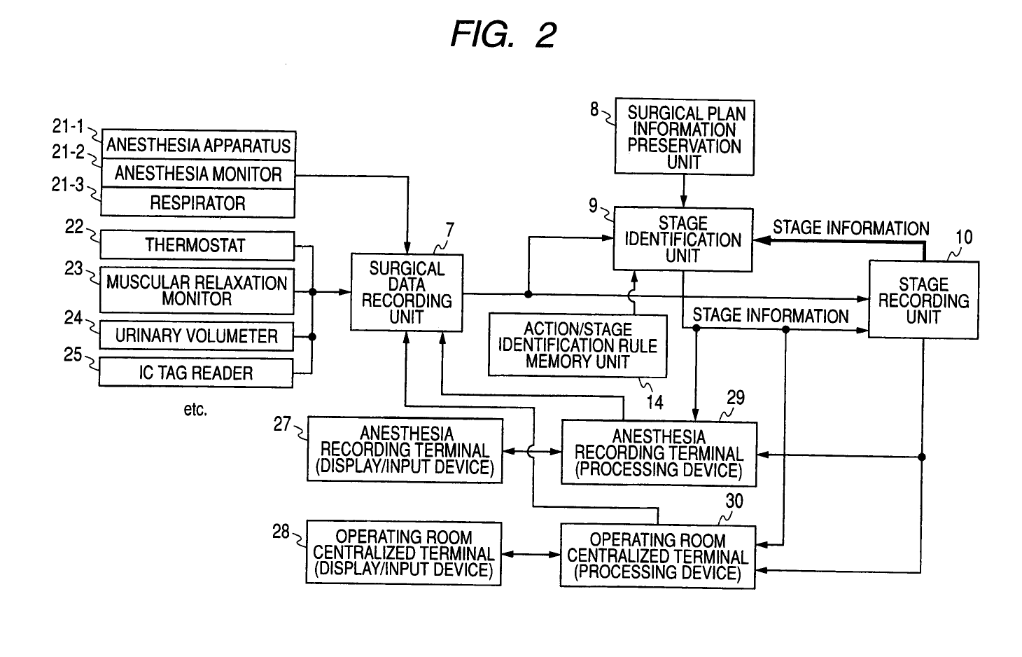

FIG. 2 shows a concrete configuration of a medical information system in accordance with the present embodiment. As the measured value acquisition unit 1, an anesthesia apparatus 21-1, an anesthesia monitor 21-1, a respirator 21-3, a thermometer 22, a muscular relaxation monitor 23, a uninary volumeter 24 are included. Moreover, as mentioned above, multiple IC tag readers 25 are included as the positional information acquisition unit 2. Incidentally, the positional information acquisition unit 2 may be defined to include the IC tag readers 25 and IC tags.

As the terminals 3, 4, and 6, a required number of terminals is disposed at required positions. For example, a display/input device 27 and a processing device 29 to be used exclusively by an anesthesiologist, and a display/input device 28 and a processing device 30 to be disposed in an operating room are included.

Medical stage identification to be performed according to the present embodiment as well as definitions of terms will be described below.

(Medical Stage)

Now, a medical stage will be described. Hereinafter, words written in double quotation marks “” shall express medical acts (actions), and words written in single quotation marks ‘’ shall express medical stages. The medical stage refers to one of processes, into which a flow of steps starting with a step of preparing for surgery and ending with a step of completing the surgery is divided, or a unit period. For example, an operation for clipping a cerebral aneurysm includes a train of medical stages ordered as mentioned below. Incidentally, each medical stage includes multiple medical acts (actions) that are correlated in order to accomplish the same object. Further, multiple medical stages may be classified into a pre-work flow, a main flow, and a post-work flow.

A train of medical stages constituting the pre-work flow includes ‘patient entry’, ‘anesthetic introduction’, ‘draping’, and ‘head immobilization’.

A train of medical stages constituting the main flow includes ‘craniotomy’, ‘search’, ‘operative field preparation’, ‘clipping’, ‘clippers check’, and ‘wound suture’.

A train of medical stages constituting the post-work flow includes ‘head release’, ‘arousal’, ‘extubation’, and ‘patient exit’.

Further, a flow may be constructed in terms of a specific purpose, for example, anesthesia.

A train of medical stages constituting an anesthesia flow includes ‘anesthetic preparation’, ‘anesthetic introduction’, ‘intubation’, ‘anesthesia sustainment’, ‘arousal’, and ‘extubation’.

The above flows concurrently progress during surgery. Consequently, multiple medical stages progress at a time during surgery. The flow is a concatenation of medical stages but does not strictly define a sequence of executing medical stages. Two medical stages included in the flow may have a time interval between them or may overlap. In a certain operation, any of medical stages scheduled as a flow may not be carried out.

The medical stage involves designation of the following pieces of information: an initiation rule, a termination rule, a warning rule, attributes of the rules, a standard value of the medical stage, items to be entered at a terminal, items to be displayed on the terminal, a layout of display on the terminal, items to be displayed on a knee board.

Incidentally, the knee board refers to a memorandum in which instruments required for executing a specific medical stage and executional precautions are written. When the knee board is created in advance, the memorandum can be referenced during surgery and utilized as auxiliary information assisting in surgery. The standard value of the medical stage refers to a standard value of the time duration of the medical stage, and is used to identify an incident.

As mentioned above, a medical stage refers to a process including multiple actions that are discriminated according to a purpose, or a period, and may be planned in advance. However, some actions cannot be planned in advance but may be performed at any time during an operative period. The actions include, for example, “aspiration”, “irrigation”, “ablation”, “hemostasis”, and “exclusion”. The flow of the actions cannot be defined as a plan in advance and cannot always be elements of a flow. However, the actions may be registered as a medical stage, and the length of the medical stage may be decided, similarly to any other medical stage, based on surgical record data.

(Medical Act (Action))

As mentioned above, a medical stage includes multiple medical acts (actions). A type of medical stage is decided based on a combination of medical acts. Moreover, the type of medical stage is decided based on a combination of medical acts and temporal relationships to preceding and succeeding medical stages that have already been decided. For identifying a medical stage, whether a specific medical act such as incision or aspiration is included may be adopted as a criterion. A medical act assisting in identifying a medical stage shall be referred to as an action.

Examples of the action are incision, aspiration, gauze insertion, exclusion, drug administration, hemostasis, and ablation.

The actions are identified based on respective criteria. For example, a type of action is decided based on at least one of the positions of medical instruments, the users of the medical instruments, the use time instants of the medical instruments, an operating state of medical equipment, biomedical information, and the position of a medical act performer. Moreover, a medical stage is identified based on whether the actions are performed, the lengths of the actions, and a time interval between adjoining actions.

(Primary Medical Stage and Secondary Medical Stage)

The medical stages are classified into primary medical stages and secondary medical stages. A medical stage constructed by modifying a certain primary medical stage shall be called a secondary medical stage. For example, a medical stage ‘clipping’ may be regarded as a primary medical stage, and a medical stage ‘test clipping’ and a medical stage ‘actual clipping’ may be registered as secondary medical stages.

The medical stage ‘clipping’ is defined as a period from the instant an operator holds clip forceps to the instant the operator releases the clip forceps. The period is finally identified as either a medical stage ‘test clipping’ or ‘actual clipping’.

The medical stage ‘test clipping’ is identified in a case where the clip forceps are closed when the operator releases the clip forceps.

The medical stage ‘actual clipping’ is identified in a case where the clip forceps are left open when the operator releases the clip forceps.

During recording within an operative period, when the clip forceps are held, the medical stage ‘clipping’ begins and a screen image registered in association with the medical stage ‘clipping’ is displayed on the terminal. Thereafter, when the clip forceps are released while being closed, the medical stage ‘clipping’ is switched to the medical stage ‘test clipping’.

In the above example, when a criterion for a secondary medical stage is satisfied, a primary medical stage is switched to the secondary medical stage. Alternatively, the primary medical stage may remain valid and the secondary medical stage may be newly constructed. When the primary medical stage is registered, actions may be able to be selected.

When display items different from those associated with a primary medical stage are designated in association with a secondary medical stage, the display on the screen complies with the contents of registration associated with the secondary medical stage.

(Terminal)

The present medical information system has multiple terminals 28 and 30 installed in an operating room. The display screen of each terminal has a full-time display area and a medical stage dependent area. Pieces of information on medical stages belonging to a specific flow are displayed in the medical stage dependent area. Pieces of information on items to be entered at a terminal, items to be displayed on the terminal, and a layout of display on the terminal are, as described previously, designated in association with a medical stage. Accordingly, a display screen image or an input screen image is presented based on the designation.

(Drafting a Surgical Plan)

For drafting a surgical plan, a surgeon enters a surgical schedule prior to surgery, for example, on a day preceding surgery. In the surgical plan preservation unit, multiple templates representative of surgical progresses are stored. Pieces of information on the templates are presented to the operator (surgeon). The operator selects an appropriate template.

For example, a template representative of cerebral aneurysm clipping A includes ‘craniotomy’, ‘search’, ‘operative field preparation’, ‘clipping’, ‘clippers check’, and ‘wound suture’.

A template representative of cerebral tumor resection (under arousal) includes ‘craniotomy’, ‘search’, ‘operative field preparation’, ‘arousal’, ‘intraoperative functional examination’, ‘incision’, ‘cytodiagnosis’, ‘intraoperative functional examination’, and ‘wound suture’.

An anesthesiologist references information on a surgical plan entered by a surgeon, and enters a plan for anesthesia. The anesthesiologist selects any of presented templates.

For example, a template representative of general anesthesia (intravenous anesthetic) includes ‘anesthetic preparation’, ‘anesthetic introduction’, ‘intubation’, ‘anesthesia sustainment’, ‘arousal’, ‘extubation’.

A template representative of general anesthesia (gas) includes ‘anesthetic preparation’, ‘anesthetic introduction’, ‘intubation’, ‘anesthesia sustainment’, ‘arousal’, and ‘extubation’.

After selecting a template, a flow is edited. For example, when an endotracheal tube is left indwelled even after the completion of surgery, a medical stage ‘extubation’ is changed to a medical stage ‘final assessment’.

Consequently, the resultant flow includes ‘anesthetic preparation’, ‘anesthetic introduction’, ‘intubation’, ‘anesthesia sustainment’, ‘arousal’, and ‘final assessment’.

Herein, the medical stage ‘final assessment’ is a kind of step including only the final assessment that used to be included in the medical stage ‘extubation’.

Both a surgeon and an anesthesiologist select a flow and edit a construction of medical stages. Thereafter, if necessary, designations concerning a criterion for initiating a medical stage, a criterion for terminating the medical stage, and a criterion for giving warning are modified. The designation modification includes modification of thresholds and modification of rules.

Further, a list of instruments required for a medical stage is added or deleted. When the list is modified, a knee board for the medical stage is reproduced and the modified list is reflected on the knee board.

(Outline of a Method of Identifying a Medical Stage)

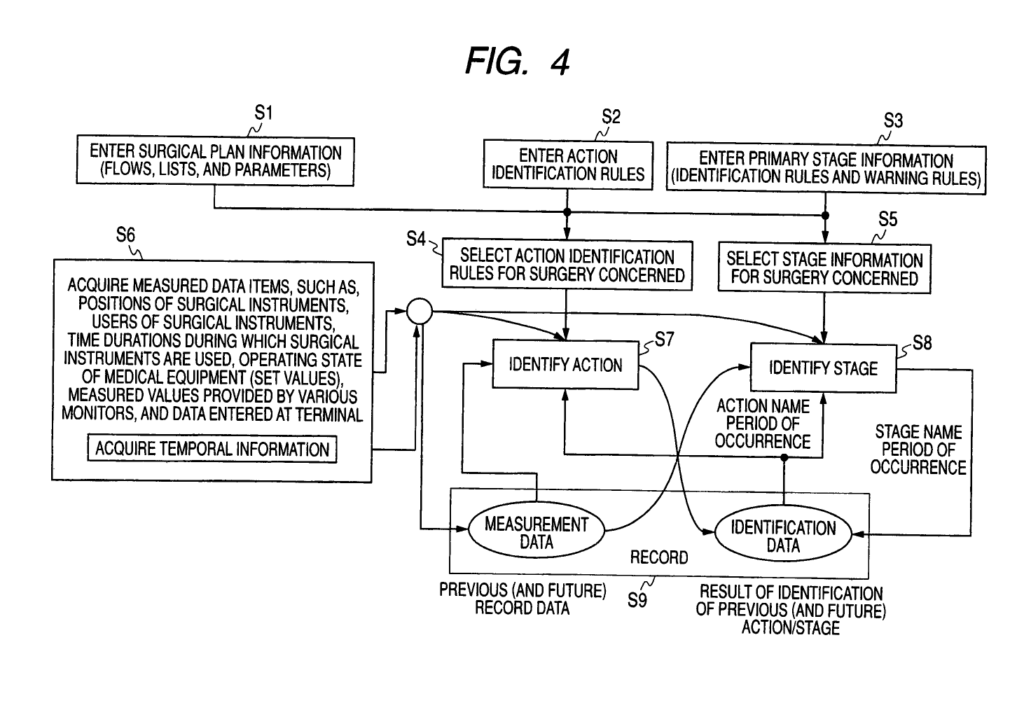

Medical stage identification is achieved according to a procedure described in FIG. 4.

At step S1, surgical plan information is supplied from the surgical plan information preservation unit 8 to the stage identification unit 9.

At steps S2 and S3, action identification rules, stage identification rules, and warning rules are supplied from the action/stage identification rule memory unit 14 to the stage identification unit 9.

At steps S4 and 5, the stage identification unit 9 selects action identification rules, which match an action scheduled to be performed according to the surgical plan, from among the action identification rules supplied from the action/stage identification rule memory unit 14. Likewise, the stage identification unit 9 selects stage identification rules and warning rules, which match medical stages specified in the surgical plan, from among the stage identification rules and warning rules supplied from the action/stage identification rule memory unit 14.

At step S6, after surgery is initiated, the measured value acquisition unit 1 repeatedly acquires the positions of surgical instruments and measured values sent from various sensors together with pieces of information on time instants of occurrence, and records them in the surgical data memory unit 7.

At step S7, for action identification, based on measured (recorded) measurement data, the stage identification unit 9 decides whether actions including aspiration and incision are performed, the time instants thereof, and the time durations thereof under the action identification rules selected at step S4. For action identification, the results of identification performed on time-sequentially preceding and succeeding actions (action names, time instants, and time durations) and pieces of information on time instants contained in the measured (recorded) data items are utilized.

At step S8, for medical stage identification, based on the results of identification performed on time-sequentially preceding and succeeding actions and medical stages (names, time instants, and durations), the measured or recorded measurement data items, and the pieces of information on time instants contained in the measurement data, the stage identification unit 9 decides the types of performed medical stages, the start time instants thereof, and the end time instants thereof (time durations) under the stage identification rules selected at step S5.

At step S9, the decided actions and medical stages are recorded in the recording unit.

What is characteristic of the present embodiment is that pieces of information on time instants included in the results of identification performed on actions and medical stages are reused to identify other actions and medical stages. What medical stage is identified based on what medical stages does not always obey a temporal sequence of occurrences of incidents.

(Identification Rules)

(Adding or Modifying an Action to be Identified)

When the usage over several months demonstrates that a method of identifying an action should be modified, identification rules can be edited. Moreover, a new action may have to be defined. In this case, a new action is added and identification rules therefor are defined.

(Adding or Modifying a Medical Stage to be Identified)

When the usage over several months demonstrates that a method of identifying an action should be modified, identification rules for a medical stage period and warning rules may be edited. Moreover, a new action may have to be defined. In this case, a new medical stage is added, and identification rules therefor are defined.

(Modifying an Action or Medical Stage Specified in a Surgical Plan)

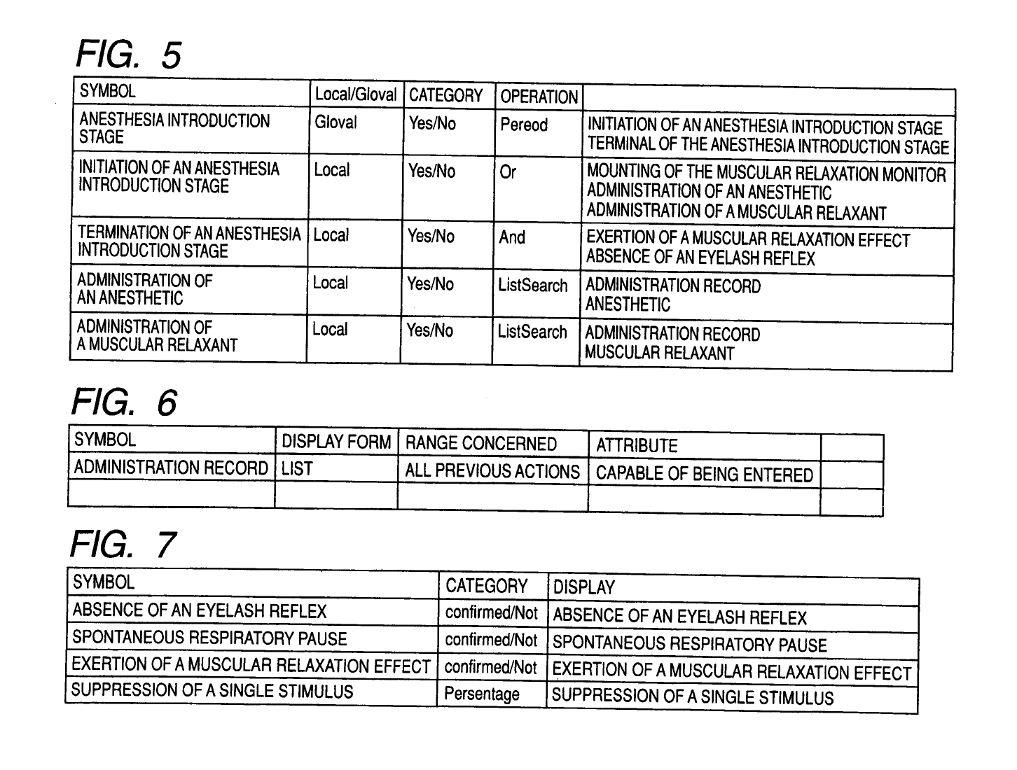

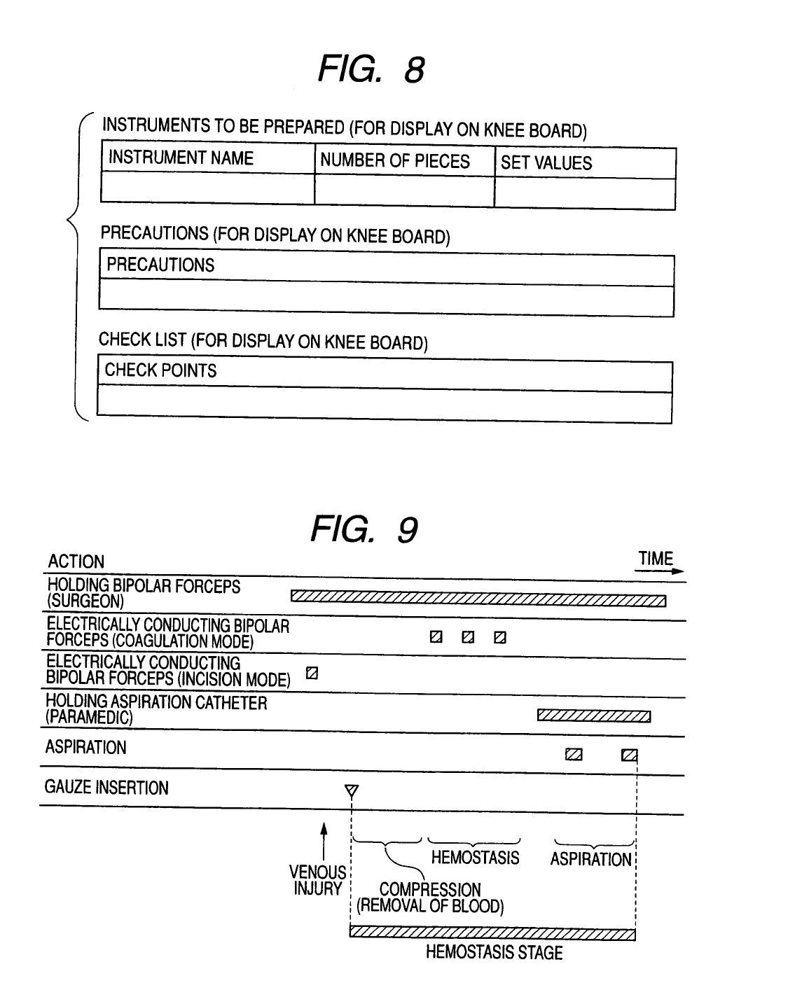

When the surgical plan unit modifies an action or a medical stage, the modification of identification rules or the like is applied to a selected operation alone. FIG. 5 lists identification rules for, for example, an anesthesia introduction stage and relevant actions. FIG. 6 lists display items associated with the anesthesia introduction stage described in FIG. 5. FIG. 7 lists items to be entered by an anesthesiologist for the purpose of deciding whether the anesthesia introduction stage is terminated (whether anesthesia is completed). FIG. 8 lists items to be displayed on a knee board in the anesthesia introduction stage described in FIG. 5.

(1) A concrete method of identifying a medical stage will be described below.

(1-1) Deciding whether a hemostasis medical stage is executed

As shown in FIG. 9, whether a hemostasis medical stage is executed is decided by checking if a surgeon holds bipolar forceps and electrically conducts the bipolar forceps in coagulation mode (operates the bipolar forces for coagulation).

(Deciding Whether the Hemostasis Medical Stage is Initiated)

Whether aspiration or gauze insertion is performed is decided by searching previous actions that have occurred over a maximum of one min since the beginning of a period during which the bipolar forceps are continuously used while being electrically conducted in coagulation mode at intervals of 30 sec or less. If the aspiration or gauze insertion is found, whether the aspiration or gauze insertion is performed is decided by searching previous actions that have performed over a maximum of one min previously from the detected aspiration or gauze insertion. This procedure is repeated to detect a time point when the aspiration or gauze insertion is performed for the first time. The time point is regarded as the start time point of the hemostasis medical stage.

(Deciding Whether the Hemostasis Medical Stage is Terminated)

Similarly to the decision made on whether the hemostasis medical stage is initiated, previous actions having occurred since the end of a period during which the bipolar forceps are continuously used while being electrically conducted in coagulation mode at intervals of 30 sec are searched in order to detect a time point when gauze insertion or aspiration is performed last at intervals of one min or less. The time point is regarded as the end time point of the hemostasis medical stage.

(Identifying Coagulation in the Hemostasis Medical Stage)

When the IC tag reader 25 attached to an operating table or worn on an operator's wrist detects an IC tag attached to bipolar forceps, “holding bipolar forceps” is decided. At the same time, who has held the bipolar forceps is recorded.

A cautery knife transmits to the recording unit 7 whether the bipolar forceps are electrically conducted and an operation mode in which the bipolar forceps are operated. A period during which the bipolar forceps are electrically conducted is checked and recorded. A period during which the bipolar forceps are electrically conducted intermittently at intervals of 30 sec or less is detected and regarded as a “hemostasis” period.

(Deciding Whether Gauze is Inserted)

An IC tag is attached to a sheet of hemostatic gauze. When the IC tag reader 25 attached to the head brace detects the IC tag, a decision is made that the gauze has been inserted into an operative field. A sensor that detects moisture on the basis of a resistance is attached to the gauze. When the sensor detects moisture and the IC tag reader attached to the head brace detects the IC tag, a decision is made that the gauze has been inserted into the operative field.

(Deciding Based on Gauze Whether Bleeding has Occurred)

Microscopic light emitting elements that emit various infrared light waves of different wavelengths and an infrared sensor are attached to gauze. A sensor that detects blood on the basis of a combination of received light waves is also attached. When the sensor detects blood, data is transmitted to the IC tag reader, and also transmitted to the acquisition unit. The data is useful in more correctly recording or deciding whether bleeding has occurred or identifying a hemostasis medical stage. Moreover, a type of blood sensor that detects light given off from a fluorescent substance which reacts to blood may be employed.

(Deciding Based on the Use Time of Gauze Whether Bleeding has Occurred)

Similarly to gauze insertion into an operative field, the timing when gauze is withdrawn from the operative field may be measured. Moreover, since different IDs are assigned to respective IC tags of respective sheets of gauze, when gauze is withdrawn from an operative field, the indwelling period during which the gauze is indwelled in the operative field can be measured. When the data is additionally utilized, a period of a hemostasis medical stage is identified as described below.

A period during which aspiration, gauze insertion, and gauze withdrawal are continuously performed without a pause of one min is detected by searching actions that have performed previously and successively to a period during which the bipolar forceps are continuously used while being electrically conducted at intervals of 30 sec or less. Among the detected periods, a period during which the number of sheets of gauze that are inserted and withdrawn is one or more or a period during which the number of sheets of gauze whose indwelling times are two min or less is one or more is regarded as a period of a medical stage ‘bleeding’.

(1-2) Deciding whether a medical stage ‘incision’ is executed

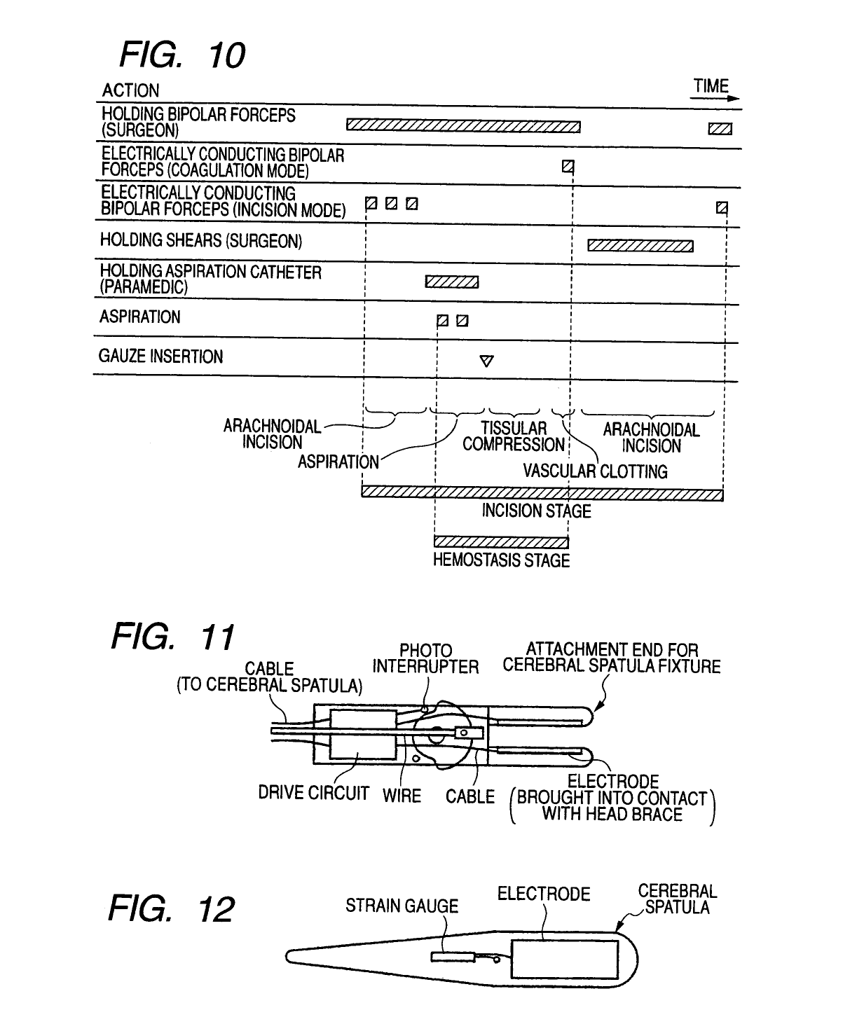

Next, referring to FIG. 10, deciding whether a medical stage ‘incision’ is executed will be described as a concrete example. A surgeon holds bipolar forceps and electrically conducts the bipolar forceps in incision mode (operates the bipolar forceps for incision).

(Identifying a Period of a Medical Stage)

A period of continuous incision shall be regarded as a period during which: the bipolar forceps are held and electrically conducted in incision mod; and incision is performed intermittently at intervals of 30 sec or less.

A period of a medical stage ‘incision’ shall be regarded as a period during which coagulation, aspiration, shears holding, and gauze insertion are performed at intervals of sixty sec or less and which precedes or succeeds the period of continuous incision. In incision, since a blood vessel is cut, the blood vessel may be clotted in advance. According to the identification method of the present embodiment, the period is recognized as a hemostasis medical stage.

(Deciding Whether Shears are Held)

A period during which an IC tag reader worn on an operator's arm detects an IC tag attached to shears shall be recognized as a period of shears holding.

(1-3) A period of a medical stage ‘aspiration’ shall be regarded as a period during which aspiration is continuously performed without a pause of 30 sec. However, the period does not include a period recognized as an irrigation medical stage to be described below.

(1-4) A period of a medical state ‘irrigation’ shall be a period during which aspiration, injection of physiological saline into an operative field, gauze insertion, and gauze withdrawal are continuously performed at intervals of 30 sec or less and during which both aspiration and injection of physiological saline to an operative field are performed.

(Injection of Physiological Saline into an Operative Field)

A syringe provided with a feature of measuring the position of a piston, a feature of transmitting a result of measurement, and an IC tag is filled with physiological saline. When an IC tag reader attached to a head brace detects the syringe and the position of the piston has shifted, a decision is made that physiological saline has been injected into an operative field. At this time, an injected amount of physiological saline is transmitted and recorded.

A method of measuring the position of a piston will be described below. (a) Piezoelectric ultrasonic transducers are incorporated in a rubber part of the distal portion of the piston, and the position of the piston is detected based on the time elapsing until ultrasonic waves originating from the ultrasonic transducers are received. (b) A rubber wheel is disposed in the vicinity of the rubber of the distal portion of the piston, and brought into contact with a syringe container. Since the wheel is rotated along with the movement of the piston, the number of rotations is measured by an encoder in order to identify a magnitude of movement. (c) A take-up encoder is used to measure the position of a cylinder. The encoder is attached to the piston, and one end of string is fixed to the syringe container.

(1-5) A medical stage ‘exclusion’ is recognized as a period during which cerebral spatula fixation is performed and during which cerebral spatula fixation and gauze insertion are continuously performed without a pause of 1 min.

(Identifying Cerebral Spatula Fixation)

Based on a cerebral spatula fixture attachment position signal, a cerebral spatula fixture/release signal, and a cerebral spatula stress signal which are concerned with a cerebral spatula fixture shown in FIG. 11, a period during which fixation of the cerebral spatula, release, and change of a stress to be imposed on the cerebral spatula (so that the stress will exceed a threshold) are performed without a pause of sixty sec with the cerebral spatula fixture attached shall be recognized as a period of cerebral spatula fixation.

When a cerebral spatula shown in FIG. 12 is fixed, or when the position of the cerebral spatula is shifted, the tension of a wire 55 included in a trunk of a retractile cerebral spatula fixture (self-retractor) is loosened so that the trunk can be deformed freely. The wire is tensed again at an appropriate position in order to lock the position. In order to tense the wire, a part coupled to the wire is rotated. A photo-interrupter used to measure a rotated position of the part is attached to a casing. The photo-interrupter detects a fixed state and a released state according to a light intercepted state. Moreover, a strain gauge is incorporated in the cerebral spatula. The strain gauge is used to measure a change in the stress to be imposed on the cerebral spatula. Signals are detected by a drive circuit, and the detected signals are transmitted to the head brace via an electrode at an end at which the fixture is attached. The head brace is provided with a receiving circuit, and the receiving circuit receives the detected signals (a cerebral spatula fixation/release signal and a cerebral spatula stress signal). Moreover, the attached position at which the cerebral spatula fixture is attached is decided. The signals are transmitted to the data acquisition unit.

(1-6) A medical stage ‘ablation’ is regarded as a period equivalent to the period of the incision medical stage in a case where the ratio of the shears holding period to the incision medical stage exceeds 20% or the number of times of shears holding is larger than the number of times of coagulation.

(2) Concrete method of identifying medical stages belonging to a main flow of surgery

An example of a flow (of cerebral aneurysm clipping) includes ‘preparation for craniotomy’, ‘craniotomy’, ‘search’, ‘relevant blood vessels ensuring’, ‘forceps insertion path ensuring’, ‘test clipping’, ‘actual clipping’, ‘clipped state check’, and ‘wound suture’.

(2-1) Rules for deciding whether a medical stage ‘craniotomy’ is initiated are that marking a skin incising region is completed and that skin incision has been initiated. Rules for deciding whether the medical stage ‘craniotomy’ is terminated are that the arrest of bleeding from a dura mater is completed, that lifting the dura mater is completed, and that checking an invasive region is completed.

Whether the rules are satisfied is decided based on the display presented after information is entered through a display screen image associated with each medical stage.

The input items typically include: whether marking a skin incising region is completed (in association with a preparation-for-craniotomy medical stage); whether skin incision is initiated; whether the arrest of bleeding from the scalp is completed; whether the arrest of bleeding from a bone is completed; whether the arrest of bleeding from a dura mater is completed; whether lifting the dura mater is completed; and whether checking an invasive region is completed.

(2-2) A rule for deciding whether a medical stage ‘search’ is initiated is that “ablation” is initiated for the first time after the completion of the medical stage ‘craniotomy’. A rule for deciding whether the medical stage ‘search’ is terminated is that recording the picture of the apex of a target aneurysm is completed.

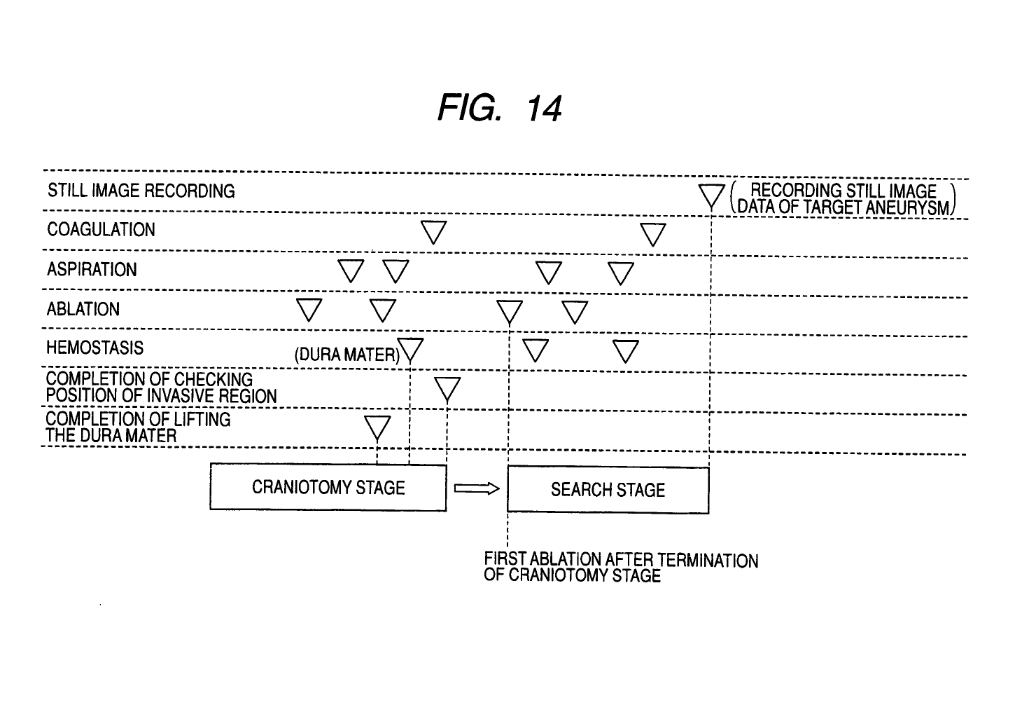

To be more specific, as shown in FIG. 14, ablation is repeated over a craniotomy stage and a search stage. From among the multiple ablations, multiple ablations performed within a specific period after the completion of the craniotomy stage are extracted. The first ablation among the extracted ablations is identified. Based on the first ablation identified, a decision is made that the search stage is initiated. Namely, the search stage is recognized under the conditions that the search stage succeeds the craniotomy stage and that ablation is performed for the first time after the completion of the craniotomy stage.

(Recording a Picture of the Apex of a Target Aneurysm)

When a target aneurysm is partly checked, a picture of an operative field is preserved as a record.

Incidentally, an aneurysm picture record button is displayed o the operating room concentrated terminals 28 and 30. A live picture visualizing an operative field is displayed on the picture display unit 28. After it is conformed that the satisfactory image quality is attained, the aneurysm picture record button is depressed. Consequently, the time instant is recorded and a still image of the operative field is recorded. The recorded still image is included in a surgical record by a surgical record summary output means after the completion of surgery. The time instant when the record button is depressed or the time instant when the still image is recorded is recognized as the end time instant of the search stage.

Moreover, when robot forceps or an endoscope is employed in surgery, it is possible to automatically decide whether the robot forceps or the tip of the endoscope has reached a scheduled position. The result of the decision can be used as one of rules for deciding whether a medical stage is terminated. First, a preoperative CT image is displayed in a surgical plan screen image prior to surgery. The position of an aneurysm to be clipped and an invasive route is marked. The robot forces or endoscope is provided with a position sensor that measures the position of the robot forceps or the direction of the distal end of the endoscope. The data acquisition unit acquires coordinates representing the position or an angle representing the direction. The information is used to calculate the position of the robot forceps or the position of the distal end of the endoscope. When the calculated position enters a range of 2 mm or less from the intersection between the border of the aneurysm and the invasive route, a decision is made that the robot forceps or endoscope has reached the aneurysm.

(Deciding Whether Warning Should be Given)

A predicted magnitude of bleeding or a reference magnitude thereof is designated. When a recorded magnitude of bleeding exceeds the predicted magnitude or reference magnitude of bleeding, warning is given. The warning is given in any medical stage other than the search medical stage. In each medical stage, a predicted magnitude or reference magnitude of bleeding is designated.

(2-3) In a medical stage ‘relevant blood vessels ensuring’, surrounding arteries are all bared in order to ensure a space, in which an occluding procedure is performed, so that the arteries can be occluded in order to temporarily cease blood flow. This is intended to quickly take measures in case bleeding occurs during clipping of an aneurysm. A rule for deciding whether a medical stage ‘relevant blood vessels ensuring’ is initiated is that the search medical stage is terminated. A rule for deciding whether the medical stage is terminated is that ensuring all relevant blood vessels is completed.

In reality, arteries that have to be ensured are registered at the time of drafting a surgical plan. Ensured picture record buttons associated with the registered arteries are displayed on the display unit of the operating room centralized terminal 28.

For example, the ensured picture record buttons include an anterior communicating artery button, a right anterior cerebral artery button, a right intermediate cerebral artery button, and a right posterior cerebral artery button.

Whether ensuring the arteries that have been registered is completed is decided as described below. After the anterior communicating artery is bared according to an actual procedure, the display screen image showing a picture record of an operative field is viewed by surgical staff members in order to verify that the anterior communicating artery is ensured satisfactorily. When the anterior communicating artery button displayed on the operating room centralized terminal is depressed, the system recognizes that ensuring the anterior communicating artery is completed. The picture of the operative field at that time is recorded.

(2-4) In a medical stage ‘clip forceps inserting path ensuring’, ablation and exclusion are intensified in order to preserve a sufficient space in which clip forceps are hung on to the neck of an aneurysm. A rule for deciding whether a medical stage ‘clip forceps inserting path ensuring’ is initiated is that ‘relevant blood vessels ensuring’ is completed or ‘test clipping’ is completed. A rule for deciding whether the medical stage is terminated is that test clipping is initiated or actual clipping is initiated. The medical stage may be repeated several times after test clipping is executed. After ‘clipped state check’ is completed, the medical stage will not be executed again.

(2-5) In a medical stage ‘test clipping’, clip forceps are actually inserted in order to decide whether clipping can be achieved without any problem. After clipping is performed, if the condition of blood flow deteriorates, the clip forceps are immediately removed. Even this case is recognized as a medical stage. A rule for deciding whether a medical stage ‘test clipping’ is initiated is that clip forceps are held. A rule for deciding whether the medical stage is terminated is that the clip forceps are released (the clip forceps are released while being closed).

(Recording an Incident)

After clipping is performed, when the clip forces are withdrawn from an operative field, it is recorded as an incident (although the clip forceps are opened in the operative field, they are returned to an instrument stage while being closed).

(Deciding Whether Clip Forceps are Held)

An IC tag is attached to the clip forceps. When an operator holds the clip forceps, the IC tag reader incorporated in the operator's wristband detects the IC tag, and decides that the clip forceps are held and the type of clip forceps.

(Identifying Actual Clipping)

A micro-switch is incorporated in the clip forceps in order to discriminate the closed state of the clip forceps (with clippers) from the open state thereof. The result of the discrimination is transmitted to the data acquisition unit via the IC tag. Whether clipping is performed is decided based on the fact that the clip forces are opened while being in an operative field (when the IC tag reader attached to the head brace detects the clip forceps, a decision is made that the clip forceps are present in the operative field).

(2-6) A medical stage ‘actual clipping’ is identical to the medical state ‘test clipping’ except the termination deciding rule. A rule for deciding whether the medical stage ‘actual clipping’ is terminated is that the clip forces are released (the clip forceps are released while being left open).

In a medical state ‘clipped state check’, blood vessels and the condition of blood flow in an aneurysm are observed (assessed) in order to verify whether they are normal. A rule for deciding whether the medical stage ‘clipped state check’ is initiated is that ‘actual clipping’ is terminated. Rules for deciding whether the medical stage is terminated are that the blood flow condition in an aneurysm is normal, that the blood flow conditions of arteries are normal, and that small blood vessels are not occluded.

(Items to be Entered for Verification)

When it is verified that the neck of the aneurysm is occluded and no blood flow is present in the aneurysm, information signifying that the blood flow condition in an aneurysm is normal is entered.

When it is verified that the arteries are pulsating and have blood flow, information signifying that the blood flow conditions of the arteries are normal is entered.

When it is verified that the small blood vessels are not occluded, information signifying that the small blood vessels are found not to be occluded as a result of checking if the small blood vessels are occluded is entered.

(3) Concrete method of identifying a medical stage concerning general anesthesia

An example of a flow includes ‘anesthetic preparation’, ‘anesthetic introduction’, ‘intubation’, ‘anesthesia sustainment’, ‘arousal’, and ‘extubation’.

(3-1) A rule for deciding whether a medical stage ‘anesthetic preparation’ is initiated is that a patient is brought into an operating room. A rule for deciding whether the medical stage is terminated is that works are all completed.

The works include: recording patient's consciousness; initiating recording of an initial electrocardiogram, a blood pressure, and an oxygen saturation; and ensuring an intravenous infusion path.

(Details of Works)

Measuring atmospheric temperature and the temperature of an operating table, bringing a patient into an operating room, recording the patient's consciousness (an anesthetic), checking the patient's name, attaching the electrodes of an electrocardiograph and checking a waveform, recording the waveform, mounting an automatic hemomanometer onto the patient and recording a measured value, mounting a pulse oximeter (artery blood oxygen saturation meter) and recording a measured value, and ensuring an intravenous infusion path using an intravenous catheter.

(Warning)

Warning is given in case: the atmospheric temperature in an operating room into which a patient is brought or the temperature of an operating table is abnormal; an ensured intravenous infusion path is abnormal; and an abnormal electrocardiogram (a patient's electrocardiogram) suggesting arrhythmia or an abnormal heart rate is obtained.

(Method of Monitoring an Intravenous Infusion Path)

An electrode is disposed at an injection site (rubber stopper) of an intravenous catheter. The electrode is connected to an electrocardiograph in order to record an intravenous infusion path electrocardiogram. When physiological saline or the like is poured, waves constituting the electrocardiogram are recorded. When the difference of the timing of an R wave included in the waves of the intravenous infusion path electrocardiogram from the timing of an R wave included in other waves falls below 5 ms, the intravenous infusion path is ensured normally. Otherwise, the intravenous infusion path is ensured abnormally.

(3-2) Rules for deciding whether a medical stage ‘anesthetic introduction’ is initiated in a case where an intravenous anesthetic is employed are that a muscular relaxation monitor is mounted and that an anesthetic is administered or a muscular relaxant is administered. Rules for deciding whether the medical stage is terminated are that an effect of muscular relaxation is exerted and that reflexes are absent. Works include: mounting the muscular relaxation monitor; administering the intravenous anesthetic; checking if a reflex is absent; administering a muscular relaxant; respirating a patient using a mask bag; waiting for two or three min; and administering another muscular relaxant unless respiration pauses.

(Warning)

Warning is given in case: a blood pressure decreases; the next medical stage begins before the medical stage is terminated; the next medical stage begins before two min elapses after a muscular relaxant is administered; and the next medical stage does not begin in five min after the muscular relaxant is administered.

(Method of Detecting Whether a Muscular Relaxation Monitor is Mounted)

A muscular relaxation monitor includes a transmitting means that utilizes infrared communication, a wireless LAN, or a wire so as to transmit a measured value (TOF count or the like). During continuous measurement, a measured value is transmitted at every measurement. When the measured value is transmitted, if the muscular relaxation monitor is mounted normally, an attribute signifying the normal mounting is appended to the measured value. The data acquisition unit receives the measured value from the muscular relaxation monitor. If the normal mounting attribute is appended, a decision is made that mounting the muscular relaxation monitor is completed.

(Detecting Exertion of a Muscular Relaxation Effect)

Exertion of a muscular relaxation effect is decided when a measured value sent from the muscular relaxation monitor falls within a range of values permitting intubation. When reaction to a cyclic stimulus is suppressed 95% or more, the muscular relaxation effect is recognized as a satisfactory level for intubation.

(Method of Detecting Intravenous Administration of a Drug)

1. A drug to be administered and a quantity thereof are entered at a terminal.

2. Prior to the administration and after the completion thereof, the weight of a syringe is measured using an electronic balance with an IC tag reader or a bar code reader. The results of measurements performed on the kind of drug, the concentration thereof, and the injected volume of thereof are transmitted to the data acquisition unit.

3. An automatic injector with a barcode reader is used to administer the drug. The injected volume of the drug is measured and transmitted. The kind of drug and the concentration thereof are recorded in an IC tag or a bar code.

(Method of Detecting Absence of a Reflex)

An eyelash reflex is a phenomenon that a person blinks when his/her eyelash is touched. An anesthesiologist actually touches a patient's eyelash and visually decides whether the reflex is present. A surgical record/analysis system 13 includes multiple terminals. The terminals include anesthetic recording terminals 27 and 29. The medical stage identification unit 9 identifies not only a medical stage relevant to anesthesia described in the present embodiment but also other multiple medical stages. The anesthetic recording terminals 27 and 29 read information on the identified anesthesia-related medical stage, produces a screen image including appropriate items to be recorded in association with an ongoing medical stage, and displays the screen image. An input item concerning absence of an eyelash reflex is shown in the screen image. When the input item is clicked, the system recognizes that an eyelash reflex is absent. Moreover, the time instant is recorded.

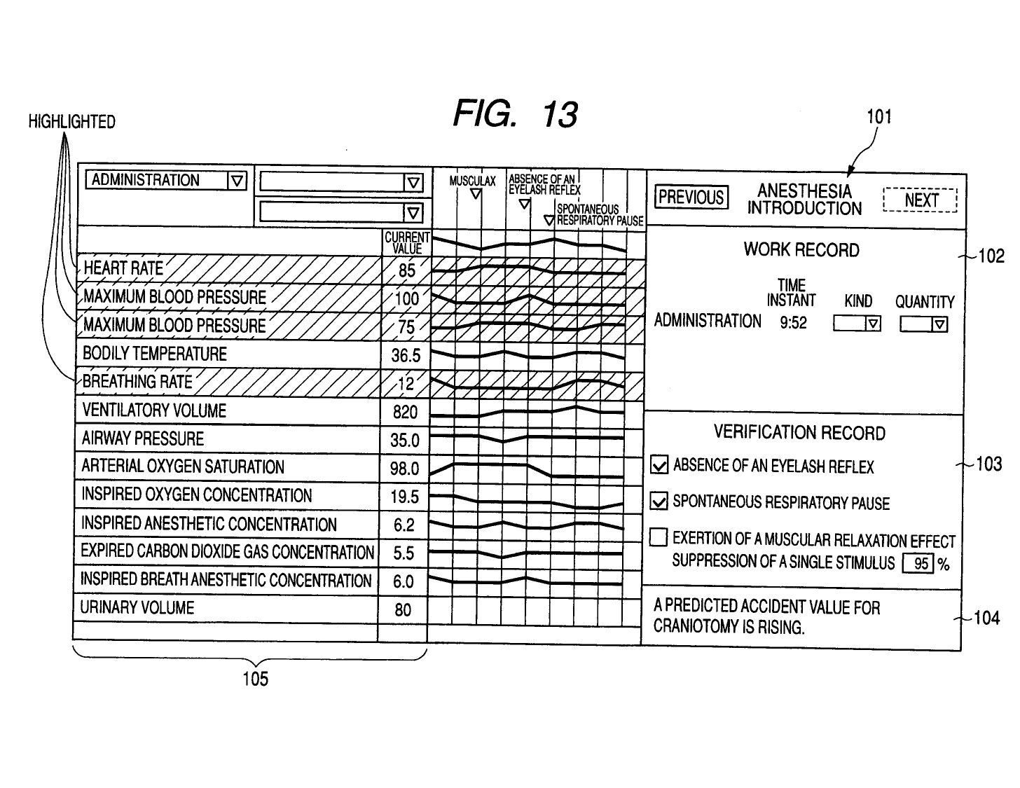

FIG. 13 shows an example of a display screen image to be displayed on the anesthetic recording terminal 27 and produced by the anesthetic recording terminal (processing device) 29. On the screen of the anesthetic recording terminal 27, a display area 101 in which a medical stage such as ‘anesthetic introduction’ is displayed is defined at the right upper section. A work record input area 102 and a verification record input area 103 in which items to be entered by a user are displayed are defined in the right middle section. An area 104 in which warning information is displayed in case a predicted value indicating occurrence of an accident and produced by the incident identification unit 11 gets high is defined in the right lower section. As the warning information, for example, a message saying that the predicted accident value for craniotomy is rising is displayed. Conceivable as a situation in which the predicted value indicating occurrence of an accident at the time of craniotomy gets higher is a situation in which an incident derived from, for example, an act of preparing an anesthetic or an act of introducing the anesthetic in a stage preceding an act of craniotomy affects the act of craniotomy.

In an area 105 defined in the left section, multiple items concerning biomedical information acquired by the measured value acquisition unit 1 and states of instruments are listed and displayed in two forms of a numerical value and an indicator representing a temporal change. The array of the multiple items is left steady. Among the displayed items, specific items associated in advance with the displayed medical stage are highlighted. Consequently, the items on which an emphasis should be put in the ongoing medical stage can be understood. Moreover, the unease of seeing attributable to the fact that the displayed positions of items are varied along with a change of medical stages can be resolved.

(3-3) Rules for deciding whether a medical stage ‘intubation’ is initiated are that a pharyngoscope is used and that a tube is used. Rules for deciding whether the medical stage is terminated are that proper ventilation is visually verified, that measuring an inspired oxygen concentration is initiated, that measuring an inspired anesthetic concentration is initiated, and that measuring an expired carbon dioxide gas concentration is initiated.

Works to be performed include: intubation; mounting an anesthetic circuit and initiating monitoring (an inspired oxygen concentration, an inspired anesthetic concentration, an expired carbon dioxide gas concentration, and an inspired breath anesthetic gas concentration); starting a respirator and initiating recording (an airway pressure, a ventilatory volume, and a ventilatory frequency); inserting a urethral catheter and initiating recording of a urinary volume (an automatic urinary volumeter); inserting a thermometer into the esophagus; and initiating measurement of an inspired oxygen concentration, an inspired anesthetic concentration, and an expired carbon dioxide gas concentration.

(Warning)

Warning is given in case any of measured values of an inspired oxygen concentration, an inspired anesthetic concentration, an expired carbon dioxide gas concentration, and an inspired breath anesthetic gas concentration is found abnormal after initiation of measurement. Warning is given in case any of measured values of a ventilatory volume and a breathing rate is found abnormal after initiation of measurement.

(Method of Deciding Whether Proper Ventilation is Visually Verified)

Similarly to detection of absence of a reflex, since an item “Visually verification of proper ventilation” is indicated on the anesthetic recording terminal, the item is clicked in order to enter what is verified and the time instant.

(3-4) A rule for deciding whether a medical stage “anesthesia sustainment” is initiated is that a medical stage “intubation” is terminated. A rule for deciding whether the medical stage is terminated is initiation of a medical stage “arousal”. During the period, multiple works (starting with laparotomy or craniotomy and ending with wound suture) are performed.

(Warning)

Warning is given in case any of measured values or values entered at the anesthetic recording terminal is abnormal. Logic for deciding abnormality or a target value (threshold) for use in deciding abnormality is designated in association with each medical stage. Whether warning is given is decided depending on an ongoing medical stage. For example, even when an abnormality in a heart rate is warned in both a medical stage ‘anesthesia sustainment’ and a medical stage ‘arousal’, different target values may be employed for the respective stages.

Warning is given in case a measured value provided by the anesthesia monitor is abnormal.

Warning is given in case a value of a ventilatory volume or a value of a breathing rate is abnormal.

Warning is given in case a heart rate, a blood pressure, or a bodily temperature varies largely.

Warning is given in case a measured value provided by the muscular relaxation monitor is abnormal.

(Displaying a Range of Target Values)

As a target value (an upper limit or a lower limit), different values may be designated for an anesthesia-related medical stage and the other medical stage (skin incision, search, synechiotomy, resection, wound suture, skin stitch, etc.) respectively. According to an identified ongoing medical stage, a target value is selected and displayed on the screen. Moreover, whether warning is given is decided based on the target value.

(3-5) A rule for deciding whether a medical stage ‘arousal’ is initiated is that supply of an inspiratory anesthetic is ceased. Rules for deciding whether the medical stage is terminated are that arousal is verified and that muscular strength is restored.

Works to be performed include: setting an inspired gas to 100% oxygen; continuing assisted respiration or controlled respiration; aspirating the trachea; checking if a tussal reflex is present; checking if spontaneous respiration occurs; administering an antagonist for muscular relaxation; checking if a swallowing reflex is present; and arousing a patient.

(Method of Deciding Whether Supply of an Inspiratory Anesthetic is Ceased)

When an anesthesia apparatus is stopped in order to cease supply of an inspiratory anesthetic, the stoppage information is transmitted to the data acquisition unit. Thereafter, when an inspired anesthetic concentration falls below a certain value, a decision is made that supply of the anesthetic is ceased.

(Method of Deciding Arousal)

Items “opening eyes”, “reacting to name calling”, “thrusting the tongue”, “opening and closing hands”, “moving toes” are displayed on the anesthetic recording terminal. When any of the items is clicked, an item “arousal verification” is displayed. When the “arousal verification” is clicked, the system decides that arousal is verified. The time instant is recorded.

(Method of Deciding Whether Muscular Strength is Restored)

Reaction is measured by the muscular relaxation monitor. After a stimulus is given four successive times at intervals of 0.5 sec, when the magnitude of the fourth reaction is 75% or more of the magnitude of the first reaction, a decision is made that muscular strength has been satisfactorily restored (to an extent that spontaneous respiration can be made).

(Warning)

Warning is given in case a heart rate, a blood pressure, or a bodily temperature falls outside the target value.

Warning is given in case a breathing rate, a ventilatory volume, or a ventilatory volume per minute falls outside the target value.

For example, when the breathing rate ranges from 8 to 25 per min and the ventilatory volume is 4 ml/kg or more, the ventilatory volume per min is 50 ml/kg or more, and a partial pressure of end-expiratory carbon dioxide is 50 mmHg, warning is given.

(3-6) As for a rule for deciding whether a medical stage ‘extubation’ is initiated during surgery, when a measured value of a cuff pressure (a difference from an ambient pressure) becomes approximately zero, a decision is made that extubation is initiated. After surgery is completed and recording is completed, when record data is browsed, whether an extubation medical stage is initiated is decided as described below. The time point of the first aspiration performed during a period during which aspiration is frequency performed by an aspirator and which precedes the timing when the cuff pressure (a difference from the ambient pressure) becomes approximately zero is recognized as the time of initiation of extubation. The period during which the aspirator is frequency used is regarded as, for example, a period during which aspiration is performed without a pause of 3 min.

Rules for deciding whether the medical stage ‘extubation’ is terminated are that supply of oxygen through an oxygen mask is initiated, that final assessment is completed, and that recording anesthetic data is completed.

Works to be performed include: opening a patient's mouth and aspirating the oral cavity and pharyngeal space while inspecting them using a pharyngoscope; deaerating the cuff by applying a weak positive pressure for the purpose of preventing suction of discharge from the pharyngeal space, and performing extubation; assessing a respiratory condition while persuading a patient to inspire 100% oxygen through a mask (auscultating both the lung fields and the trachea); removing a nasogastric tube while aspirating it, if necessary; observing the patient for at least five min after extubation, and ceasing supply of oxygen if there is no problem or not ceasing the supply; assessing the patient's arousal and others, and completing anesthetic recording; transporting the patient to the exit of the operating room supposing there is no problem, and contacting a person in charge of taking care of the patient.

(Deciding that a Cuff Pressure is Approximately Zero)

As for the cuff pressure (a difference from the ambient pressure), a barometer is disposed at the cuff air inlet attached to the endotracheal tube. A difference between the pressure in the cuff and the atmospheric pressure is measured, and the measured pressure is displayed and transmitted to the data acquisition unit. The thus measured pressure includes an abnormal measured value derived from noise or shock. An abrupt change is detected. Abnormal values representing the abrupt change are removed, and the remaining values are used for interpolation. Thereafter, a wideband filter is used to minimize the noise. Whether the cuff pressure is approximately zero is decided by checking if an obtained wave falls within a predefined range.

(Method of Deciding Whether an Aspirator is in Use)

(1) When the IC tag reader attached to an operating table or worn on an operator's wrist detects an IC tag attached to an aspiratory catheter, a decision is made that “the aspiratory catheter is held”. At the same time, who is holding the aspiratory catheter is recorded.

(2) When an atmospheric pressure sensor attached to a suction flask reads an atmospheric pressure identical to the atmospheric pressure in the environment, the aspirator is decided to be unused. When a certain negative pressure persists, the aspirator is decided to be standby. When an intermittent variation in the atmospheric pressure is detected among negative pressures larger than the negative pressure that persists when the aspirator is standby, the aspirator is decided to be in operation. When the atmospheric pressure varies multiple times at intervals of a certain time (for example, ten sec) or less, a period during which a series of variations is detected in the atmospheric pressure is regarded as one aspiration time.

(Method of Deciding Whether Oxygen is Supplied Through an Oxygen Mask)

A sensor for measuring a tension or the length of a band is attached to the band of an oxygen mask. A take-up rotary encoder is used to measure the length of the band. A stretchable tube through which measurement string is passed is attached to the band. Moreover, a flowmeter (for example, windmill) is attached to an oxygen tube attachment portion of the mask in order to measure the flow rate of a gas. The measured values are processed by a circuit incorporated in the mask, and transmitted to the data acquisition unit. Whether oxygen is supplied is decided by checking the band length and flow rate. When the band length is equal to or larger than a certain value and the flow rate is abnormal, a decision is made that oxygen is supplied. The deciding means may be implemented by the circuit incorporated in the mask or by the data acquisition unit (or the other device included in the recording system).

(Deciding Whether Assessment of Arousal is Completed)

The same method as the one adopted in the arousal medical stage is adopted to decide whether a patient is aroused. When the patient leaves an operating room, or after anesthetic recording is completed, arousal is assessed in the same manner and recorded. Since items to be entered relative to vital data items (a pulse rate, a breathing rate, a bodily temperature, and a blood pressure), a respiratory condition, a complexion, and presence or absence of a pain or nausea and vomiting are displayed, the results of verification are entered.

(Deciding Whether Recording of Anesthetic Data is Completed)

Since items concerning a used gas volume, a total volume of infusion, a total volume of transfusion, a urinary volume, and a total blood loss are displayed, the contents of the items are verified. A Verify button is then depressed.

Incidentally, the present invention is not limited to the aforesaid embodiment. In practice, the constituent features of the invention may be modified without a departure from the gist of the invention. Moreover, multiple constituent features disclosed in relation to the embodiment may be properly combined in order to construct various inventions. For example, some of the constituent features included in the embodiment may be excluded. Moreover, the constituent features of different embodiments may be combined appropriately.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical information system comprising:
a measurement unit that measures at least the positions of medical instruments and biomedical information;
a medical act identification unit that decides the types of performed medical acts and the time instants of performance, at which the medical acts are performed, on the basis of the measured positions of the medical instruments and the measured biomedical information; and
a medical stage identification unit that decides the types of medical stages, into which a flow of steps starting with a step of preparing for surgery and ending with a step of completing the surgery is divided in terms of the purpose of work, and the time instants of the medical stages on the basis of the decided types of medical acts, the decided time instants of performance, the other types of medical stages that have already been decided, and the time instants of the other types of medical stages that have already been decided.

2. The medical information system according to claim 1, further comprising a recording unit that records the decided types of medical acts, the decided time instants of performance, the decided types of medical stages, and the decided time instants of the medical stages.

3. The medical information system according to claim 1, wherein the medical stage identification unit decides the types of medical stages and the time instants thereof on the basis of the decided types of medical acts and the time instants thereof, the other types of medical stages and the time instants thereof, and a planned procedure of medical stages.

4. The medical information system according to claim 1, further comprising a memory unit in which rules for identifying the medical stages are stored.

5. The medical information system according to claim 1, further comprising a display unit on which multiple items concerning the decided types of medical stages and the biomedical information are displayed in the form of a numerical value or an indicator representing a temporal change.

6. The medical information system according to claim 5, wherein the display unit changes the items to be displayed according to an identified medical stage.

7. The medical information system according to claim 5, wherein the display unit displays in a highlighted manner specific items out of the plurality of items to be displayed, and changes the specific items from ones to others according to an identified medical stage.

8. A medical information system that records data items concerning medical acts, comprising:
a recording unit that records a signal sent from medical equipment and a signal sent from a measuring instrument;
a medical act identification unit that decides the types of performed medical acts and the time instants of performance, at which the medical acts are performed, on the basis of the recorded signal sent from the medical equipment and the recorded signal sent from the measuring instrument; and
a medical stage identification unit that extracts at least one medical act, which is performed during a specific period, from among the plurality of decided types of medical acts, and decides the types of medical stages, into which a flow of steps starting with a step of preparing for surgery and ending with a step of completing the surgery is divided in terms of the purpose of work, and the time instants of the medical stages on the basis of the extracted medical act.

9. The medical information system according to claim 8, further comprising a recording unit that records the decided types of medical acts, the decided time instants of performance, the decided types of medical stages, and the decided time instants of the medical stages.

10. The medical information system according to claim 8, wherein the medical stage identification unit decides the types of medical stages and the time instants thereof on the basis of the decided types of medical acts and the time instants thereof, the other types of medical stages and the time instants thereof, and a planned procedure of medical stages.

11. The medical information system according to claim 8, further comprising a memory unit in which rules for identifying the medical stages are stored.

12. The medical information system according to claim 8, further comprising a display unit on which a plurality of items concerning the decided types of medical stages and the biomedical information is displayed in the form of a numerical value or an indicator representing a temporal change.

13. The medical information system according to claim 12, wherein the display unit changes items to be displayed according to a decided type of medical stage.

14. The medical information system according to claim 12, wherein the display unit displays in a highlighted manner specific items out of the plurality of items to be displayed, and changes the specific items from ones to others according to a decided type of medical stage.

15. The medical information system according to claim 8, further comprising:
a memory unit in which data items of rules for identifying the medical stages and medical acts are stored; and
a reading unit that reads the data of an identification rule from the memory unit, wherein:
the medical stage identification unit decides the time instant of a medical stage relating to a medical stage, which has already been identified, on the basis of the time instants of medical acts performed during a specific period stipulated in an identification rule read from the memory unit.
